(19) United States
(12) Patent Application Publication
(10) Pub. No.: US 2023/0190883 A1
(43) Pub. Date: Jun. 22, 2023

(54) STABLE PEPTIDES HAVING RENALASE AGONIST ACTIVITY

(71) Applicant: Bessor Pharma, LLC, Framingham, MA (US)

(72) Inventors: Gary Desir, Framingham, MA (US); Arthur Felix, Framingham, MA (US); Wallace Dairman, Framingham, MA (US); Anthony Barrett, Rio de Janeiro (BR); Barry Berkowitz, Framingham, MA (US)

(21) Appl. No.: 17/928,381

(22) PCT Filed: May 28, 2021
(86) PCT No.: PCT/US2021/034706

§ 371 Date: Nov. 29, 2022

Related U.S. Application Data
(60) Provisional application No. 63/032,055, filed on May 29, 2020.

(51) Int. Cl. A61K 38/44; C12N 9/02; A61P 13/12; A61P 1/18

(52) U.S. Cl. CPC A61K 38/44; C12N 9/0036; C12Y 106/03; A61P 13/12; A61P 1/18

(57) ABSTRACT

Disclosed are stable peptides that have renalase agonist activity and are useful for treating diseases such as AKI and AP, including those relating to SARS-CoV-2

### REFERENCE TO RELATED APPLICATIONS

This application is a National Stage application of International Patent Application No. PCT/US2021/034706, filed on May 28, 2021, which claims priority to U.S. Provisional Patent Application No. 63/032,055, filed on May 29, 2020, each of which is hereby incorporated by reference in its entirety.

### STATEMENT REGARDING FEDERALLY SPONSORED RESEARCH OR DEVELOPMENT

This invention was made with government support under grant number R44DK111251, awarded by the National Institute of Diabetes and Digestive and Kidney Diseases of the National Institutes of Health. The government has certain rights in the invention.

### REFERENCE TO SEQUENCE LISTING SUBMITTED VIA EFS-WEB

The contents of the ASCII text file of the sequence listing named “8712-0001WO SEQUENCE LISTING_seq_ST25”, which is 70 kb in size, was created on May 21, 2021, and electronically submitted via EFS-Web with the application, is incorporated herein by reference in its entirety.

### FIELD OF THE INVENTION

The present disclosure relates to stable peptides that exhibit tissue protective activity and are useful for treating renal or pancreatic disease such as acute kidney injury or acute pancreatic injury, particularly renal disease or pancreatic Injury associated or exacerbated with SARS-CoV-2.

### BACKGROUND OF THE INVENTION

The following discussion is provided merely to aid the reader in understanding the disclosure and is not admitted to describe or constitute prior art thereto.

Renalase (“RNLS”) is secreted by the kidney and has multiple biological functions. See, for example, U.S. Pat. Nos. 7,700,095; 7,858,084; 7,932,067; and 10,066,025; and PCT patent application Ser. No. 18/67608, which are incorporated herein in their entirety as if fully reproduced herein. Administration of recombinant or biologically-isolated renalase has been shown to treat certain diseases and conditions, as described in the above-mentioned United States patent 10,066,025 and in Wang, et al. J. Am. Soc. Nephrol. 2014 Jun; 25(6): 1226-1235. It has been shown RNLS prevents acute kidney injury independent of its oxidase function by a cell signaling mechanism (Wang et al 2014).

Acute kidney injury (AKI) and acute pancreatitis (AP) are seen at least ⅓^{rd} and ⅕^{th} of hospitalized COVID-19 patients, respectively, and occur more often with severe disease. Renalase (RNLS) is a unique circulating protein that potently increases cell survival and reduces inflammation to treat renal diseases such as AKI and/or AP.

However, synthesis and administration of the entire renalase chain A is cumbersome and expensive and may convey complex pharmacologic properties from both the oxidase function as well as the cell signaling function (Wang et al, 2014). Stable synthetic peptides exhibiting renalase agonist activity particularly with the cell signaling tissue repair function would be highly desirable. The present disclosure provides such peptides.

### SUMMARY OF THE INVENTION

The present invention relates to novel peptides derived from Renalase Chain A (1-342) (SEQ ID NO: 1) shown in FIG. 1. The peptides comprise at least residues 220 to 229 of Renalase A (1-342) in which Cys²²⁰ is replaced by the residue of an amino acid selected from:

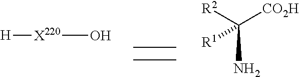

wherein R¹ and R² are independently H; C₁ to C₈ n-alkyl optionally substituted by hydroxyl; C3 to C8 branched alkyl optionally substituted by hydroxyl; C₄ to C₈ doubly branched alkyl optionally substituted by hydroxyl; C₃ to C₆ cycloalkyl optionally substituted by hydroxyl or methyl or both at any position or positions and which includes all structurally feasible stereoisomeric entities; CH₂—C₃ to C₆ cycloalkyl optionally substituted by hydroxyl or methyl or both at any position or positions and R¹ and R² may be linked together as (CH₂)ₙ optionally substituted at any position by methyl or hydroxyl or both, wherein n is 2, 3, 4, or 5; and

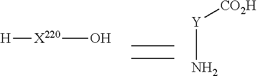

wherein Y is (CH₂)ₙ optionally substituted at any position or positions by methyl or hydroxyl or both, provided that the carbon bearing the amino group may be substituted only by methyl, wherein n is 2, 3, 4, 5; cis- or trans-1,2-cyclopropanediyl, cis- or trans-1,2-cyclobutanediyl, cis- or trans-1,3-cyclobutanediyl, cis- or trans-1,2-cyclopentanediyl, cis- or trans-1,3-cyclopentanediyl, cis- or trans-1,2-cyclohexanediyl, cis- or trans-1,3-cyclohexanediyl, or cis- or trans-1,4-cyclohexanediyl optionally substituted by at any position or positions by methyl or hydroxyl or both provided that the carbon bearing the amino group may be substituted only by methyl and which includes all structurally feasible diastereoisomeric entities.

This modification improves the biological potency of the peptides and stabilizes them from potential in situ dimerization and/or oligomerization. Preferably, X²²⁰ is selected from Ser, Ala, Leu, Val, Ile, Nle, β-Ala, Aib, cyclopropyl-glycine, and (cyclopropylmethyl)-glycine.

Additional amino acid residues may be added at the NH₂-terminus of X²²⁰ corresponding to some or all of the amino acid residues in sequence from positions 205 to 219 of renalase A shown in the corresponding positions of FIG. 1 or at the COOH-terminus of Lys²²⁹ corresponding to some or all of the amino acid residues in sequence from positions 230 to 253 of renalase A shown in the corresponding positions of FIG. 1. In addition, long chains of poly(ethylene)glycol [PEG] or bis-poly(ethylene)glycol (bis-PEG) of varying average molecular weight, for example, between 5000 and 20,000 amu, or similar polymers known in the art (as described below) may be attached to either the NH₂-terminus of X²²⁰ or to the NH₂-terminus of longer fragments or to the COOH-terminus of Lys²²⁹ or to the COOH-terminus of longer fragments.

The expression “corresponding to some or all of the amino acid residues in sequence from positions 205 to 219 of renalase A” means the added amino acid residues may comprise in sequence the residues from position 219, from positions 218 and 219, from positions 217 through 219, from positions 216 through 219, and so on up to from positions 201 through 219. Similarly, the expression ^{—}corresponding to some or all of the amino acid residues in sequence from positions 230 to 253 of renalase A″ means the added amino acid residues may comprise in sequence the residues from position 230, from positions 230 to 231, from positions 230 through 232, and so on up to from positions 230 through 253.

The present disclosure also provides methods of treating renal diseases, including AKI and AP and renal diseases resulting from SARS-CoV-2.

### BRIEF DESCRIPTION OF THE DRAWINGS

FIG. 1 depicts Renalase Chain A (1-342).

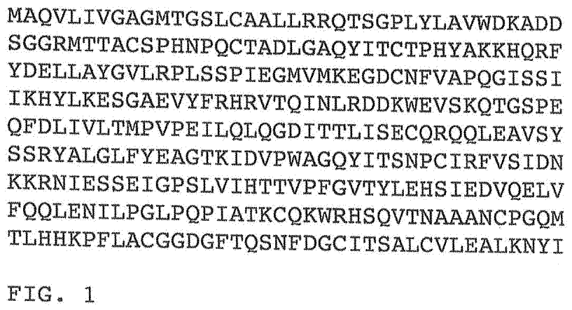

FIG. 2 show renalase agonist peptide 10 (BP 1002) reduces injury on a mouse model of acute pancreatitis.

FIG. 3 shows activity of renalase agonists against cisplatin lethality.

FIG. 4 shows renalase agonist peptide 81 reduced cisplatin induced kidney injury.

FIG. 5 shows renalase agonist BP-1002 decreases inflammatory response to SARS-CoV-2 peptides.

FIG. 6 shows the anti-inflammatory effect of peptide 10 (BP 1002).

FIG. 7 shows another presentation of the anti-inflammatory effect of peptide 10.

FIG. 8 shows the anti-inflammatory effect of peptide 81.

FIG. 9 shows the comparative anti-inflammatory effect of peptide 10 (BP 1002).

FIG. 10 shows the correlation between decreased renalase levels in COVID-19 patients and mortality.

FIG. 11 shows the renalase agonist peptide 10 (BP-1002) improves survival in a COVID-19 mouse model.

FIG. 12 shows treatment with the renalase agonist, peptide 10, (BP-1002) protects renalase-deficient mice in a model of severe viral infection.

FIG. 13 shows peptide 10, (BP-1002) was effective in reducing necrosis in a model of cerulin-induced severe pancreatitis.

FIG. 14 shows peptide 10, (BP-1002) decreases levels of cronin 1, a marker for inflammatory cells.

### DETAILED DESCRIPTION OF THE INVENTION

In accordance with the present disclosure are provided synthetic peptides, pharmaceutical compositions comprising the peptides, and methods of treatment of human disease using the synthetic peptides and pharmaceutical compositions.

The inventors have discovered that certain peptide fragments within the sequence of renalase chain A exhibit renalase agonist activity. However, they have also discovered these peptides are unstable and therefore unsuitable for use as a therapeutic. They have discovered that the instability of the peptide fragments can be eliminated by replacing the cysteine residue at position 220 of Renalase Chain A (1-342) by a different appropriately selected amino acid. In addition, the modified peptide fragments have increased potency as compared to unmodified fragments.

The amino acid at position 220 may be selected from the residue of an amino acid selected from:

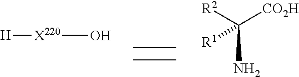

wherein R¹ and R² are independently H; C₁ to C₈ n-alkyl optionally substituted by hydroxyl; C3 to C8 branched alkyl optionally substituted by hydroxyl; C₄ to C₈ doubly branched alkyl optionally substituted by hydroxyl; C₃ to C₆ cycloalkyl optionally substituted by hydroxyl or methyl or both at any position or positions and which includes all structurally feasible stereoisomeric entities; CH₂—C₃ to C₆ cycloalkyl optionally substituted by hydroxyl or methyl or both at any position or positions and R¹ and R² may be linked together as (CH₂)ₙ optionally substituted at any position by methyl or hydroxyl or both, wherein n is 2, 3, 4, or 5; and

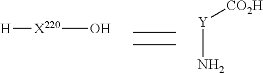

wherein Y is (CH2)ₙ optionally substituted at any position or positions by methyl or hydroxyl or both, provided that the carbon bearing the amino group may be substituted only by methyl, wherein n is 2, 3, 4, 5; cis- or trans-1,2-cyclopropanediyl, cis- or trans-1,2-cyclobutanediyl, cis- or trans-1,3-cyclobutanediyl, cis- or trans-1,2-cyclopentanediyl, cis- or trans-1,3-cyclopentanediyl, cis- or trans-1,2-cyclohexanediyl, cis- or trans-1,3-cyclohexanediyl, or cis- or trans-1,4-cyclohexanediyl optionally substituted by at any position or positions by methyl or hydroxyl or both provided that the carbon bearing the amino group may be substituted only by methyl and which includes all structurally feasible diastereoisomeric entities.

The peptides of the invention comprise Renalase A fragments including positions 220-229 with X²²⁰ as described above and comprising additional amino acids at the appropriate positions of Renalase A on one or either side of positions 220-229, the peptides generally varying in length from about 49 amino acids to about 20 amino acid residues, although they may be longer as desired.

Additionally, poly(ethylene)glycol (PEG) or bis-poly(ethylene)glycol (Bis-PEG) of various average molecular weights (e.g., between 500 and 20,000 amu) may be attached to either terminus of the peptide to promote longer-term activity of the peptide, as is well-known in the pharmaceutical art. Poly(ethylene)glycol is an amphiphilic polymer consisting of repeating units of ethylene oxide which may be assembled in linear or branched structures to give a range of PEGs with different configurations and molecular weights. PEG must be activated in order to be covalently conjugated to appropriate sites in biopharmaceutical compounds (including peptides and proteins) thereby improving their pharmacologic and pharmaceutical properties. In addition to improving solubility, conjugation with PEG can protect the biopharmaceutical compound from the host's immune system thereby reducing immunogenicity and antigenicity and prolonging biological half-life. The resulting PEGylated pharmaceuticals may be used at reduced dosage and frequency without diminished efficacy. PEG is available in a variety of average molecular weights and may be functionalized at one end (the other end is usually protected as the methoxy). Alternatively, both ends of the polymer may be functionalized resulting in homobifunctional or heterobifunctional derivatives which can be used for linking two entities. A review of both first- and second-generation PEG derivatives, with more varied and efficient functional groups for conjugation to peptides and proteins has been described by Roberts, Adv. Drug Deliv. Rev.,54, 459 (2002). Third generation PEGylating agents have been developed where the polymer has been branched and may offer additional advantages for protecting proteins from proteolysis and further reducing immunogenicity and antigenicity. Although [PEG] is commonly used for conjugation to peptides and proteins, other suitably functionalized polymers, including, but not limited to carbohydrate moieties, that have appropriate flexibility can also be used and has been reviewed by Sola and Griebenow, J. Pharm. Sci., 98, 1223 (2009) and by Witteloostuijn, Pedersen, and Jensen, ChemMedChem, 11, 2474 (2016), both of which are incorporated herein by reference.

Selected amino acids for substitution at position 220 of the peptides of the invention include glycine, serine, alanine, leucine, valine, isoleucine, norleucine, beta-alanine, cyclopropyl-glycine, (cyclopropylmethyl)-glycine, and other hydrophobic amino acids as well as the D-amino acid enantiomers of the described amino acids. Additionally, the Lys²²⁹ may be replaced with its D-isomer to afford additional stability. Other amino acids, including Lys²⁰⁵, Arg²²², Lys²³⁰ and/or Arg²³¹ may also be replaced with the corresponding D-amino acids for similar enhancement of stability to enzymatic degradation.

In one embodiment, there are provided peptides comprising R-X²²⁰-Ile²²¹-Arg²²²-Phe²²³-Val²³⁴-Ser²²⁵-Ile²²⁶-Asp²²⁷-Asn²²⁸-Lys²²⁹-R′ wherein the superscripts represent positions within the renalase A chain (1-342), R is selected from Ac-Ala-Gly-Thr-, Ac-Gly-Thr-, Ac-Thr—, Ac- , Ac-Z, H-, H-Z, B-Z- and B, wherein Z is selected from one or more of the amino acid residues at positions 205-219 of the renalase A chain, R′ is selected from —NH₂, Z′-NH₂, -B, and Z′-B, wherein Z′ is selected from one or more of the amino acid residues at positions 230-253 of the renalase A chain, B is PEG or bis-PEG, and X is the residue of an amino acid selected from:

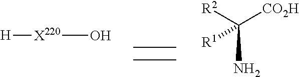

wherein R¹ and R² are independently H; C₁ to C₈ n-alkyl optionally substituted by hydroxyl; C3 to C8 branched alkyl optionally substituted by hydroxyl; C₄ to C₈ doubly branched alkyl optionally substituted by hydroxyl; C₃ to C₆ cycloalkyl optionally substituted by hydroxyl or methyl or both at any position or positions; CH₂—C₃ to C₆ cycloalkyl optionally substituted by hydroxyl or methyl or both at any position or positions and R¹ and R² may be linked together as (CH₂)ₙ optionally substituted at any position by methyl or hydroxyl or both, wherein n is 2, 3, 4, or 5; and

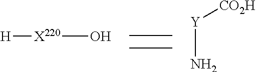

wherein Y is (CH₂)ₙ optionally substituted at any position or positions by methyl or hydroxyl or both, provided that the carbon bearing the amino group may be substituted only by methyl, wherein n is 2, 3, 4, 5; 1,2-cyclopropanediyl, 1,2-cyclobutanediyl, 1,3-cyclobutanediyl, 1,2-cyclopentanediyl, 1,3-cyclopentanediyl, 1,2-cyclohexanediyl, 1,3-cyclohexanediyl, or 1,4-cyclohexanediyl optionally substituted by at any position or positions by methyl or hydroxyl or both, provided that the carbon bearing the amino group may be substituted only by methyl and provided that the peptide comprises at least 20 amino acid residues.

In another embodiment are provided peptides having the sequence R-Lys²⁰⁵ -Ile²⁰⁶-Asp²⁰⁷-Val²⁰⁸-Pro²⁰⁹-Trp²¹⁰-Ala²¹¹-Gly²¹²-Gln²¹³-Tyr²¹⁴-Ile²¹⁵-Thr²¹⁶-Ser²¹⁷-Asn²¹⁸-Pro²¹⁹-X²²⁰-Ile²²¹-Arg²²²-Phe²²³-Val²³⁴-Ser²²⁵-Ile²²⁶-ASP²²⁷-Asn²²⁸-Lys²²⁹-Lys²³⁰-Arg²³¹-Asn²³²-Ile²³³-Glu²³⁴-Ser²³⁵-Ser²³⁶-Glu²³⁷-Ile²³⁸-Gly²³⁹-Pro²⁴⁰-Ser²⁴¹ -Leu²⁴²-Val²⁴³-Ile²⁴⁴-His²⁴⁵-Thr²⁴⁶-Thr²⁴⁷-Val²⁴⁸-Pro²⁴⁰-Phe²⁵⁰-Gly²⁵¹-Val²⁵²-Thr²⁵³-R′ wherein R is selected from Ac-Ala-Gly-Thr-, Ac-Gly-Thr-, Ac-Thr—, Ac-, H-, and PEG, R′ is selected from -Tyr-Leu-Glu-NH₂, Tyr-Leu-NH₂, -Tyr-NH₂, -NH₂, -OH, and PEG, and X²²⁰ is selected from Gly, Ser, Ala, Leu, Val, Ile, Nle, 6-Ala cyclopropyl-Gyl, (cyclopropylmethyl)-Gly, and Aib.

Thus, peptide of the invention include: [X²²⁰]-Ac-Renalase A (205-240)-NH₂ (SEQ ID NO: 2), [X²²⁰]-Ac-Renalase A (214-253)-NH₂, (SEQ ID NO: 3) [X²²⁰]-Ac-Renalase A (214-240)-NH₂ (SEQ ID NO: 4), and [X²²⁰]-Ac-Renalase A (205-253)-NH₂ (SEQ ID NO: 5), wherein X is selected from glycine, serine, alanine, leucine, valine, isoleucine, norleucine, cyclopropyl-glycine, (cyclopropylmethyl)-glycine, and beta-alanine.

Representative compounds of the present invention include but are not limited to:

[Ala²²⁰]-Ac-Renalase A (205-240)-NH₂ (SEQ ID NO: 6)

[Ala²²⁰]-Ac-Renalase A (214-253)-NH₂ (SEQ ID NO: 7)

[Ala²²⁰]-Ac-Renalase A (214-240)-NH₂ (SEQ ID NO: 8)

[Ala²²⁰]-Ac-Renalase A (205-253)-NH₂ (SEQ ID NO: 9)

[Val²²⁹]-Ac-Renalase A (214-240)-NH₂ (SEQ ID NO: 10)

[Ser²²⁹]-Ac-Renalase A (214-253)-NH₂ (SEQ ID NO: 11)

[Ala²²⁰, D-Lys²²⁹]-Ac-Renalase A (205-240)-NH₂ (SEQ ID NO: 12)

[Ser²²⁰]-Ac-Renalase A (205-240)-NH₂ (SEQ ID NO: 13)

[Ala²²⁰]-Ac-Renalase A (214-234)-NH₂ (SEQ ID NO: 14)

[Ala²²⁰]-Ac-Renalase A (220-239)-NH₂ (SEQ ID NO: 15)

[Gly²²⁰]-Ac-Renalase A (205-240)-NH₂ (SEQ ID NO: 16)

[Gly²²⁰]-Ac-Renalase A (214-253)-NH₂ (SEQ ID NO: 17)

[cyclopropyl-Gly²²⁰]-Ac-Renalase A (205-253)-NH₂ (SEQ ID NO: 18)

[(cyclopropylmethyl)-Gly²²⁰]-Ac-Renalase A (214-240)-NH₂ (SEQ ID NO: 19)

One representative example of a PEGylated peptide of the invention is [bis-PEG5000]- Lys²⁰⁵-Ile²⁰⁶-Asp²⁰⁷-Val²⁰⁸-Pro²⁰⁹-Trp²¹⁰-Ala²¹¹-Gly²¹²-Gln²¹³-Tyr²¹⁴-Ile²¹⁵-Thr²¹⁶-Ser²¹⁷-Asn²¹⁸-Pro²¹⁹-Ala²²⁰-Ile²²¹-Arg²²²-Phe²²³-Val²³⁴-Ser²²⁵-Ile²²⁶-Asp²²⁷-Asn²²⁸-Lys²²⁹-Lys²³⁰-Arg²³¹-Asn²³²-Ile²³³-Glu²³⁴-Ser²³⁵-Ser²³⁶-Glu²³⁷-Ile²³⁸-Gly²³⁹-Pro²⁴⁰-NH₂. (SEQ ID NO: 20)

Peptides are generally prepared using solid phase synthesis, such as that described by Merrifield, J. Am. Chem. Soc., 85, 2149 (1963) although other equivalent chemical syntheses known to one of ordinary skill may be used, including liquid-phase synthesis or production biologically using recombinant technologies. Solid phase synthesis is commenced from the C-terminal end of the peptide by coupling an NH₂-protected amino acid to a suitable resin. The starting material is prepared by attaching the COOH-terminal of N^{alpha}-9-fluorenylmethoxycarbonyl (Fmoc) amino acid to commercially available 4,4′-dimethoxybenzhydryl-amine, (Mbh)-handle, that is linked to a solid phase resin. The solid phase syntheses and coupling with Fmoc-amino acids (including suitably protected side chains for trifunctional amino acids) proceeded using a carbodiimide/HOBt mediated reaction in a stepwise elongation of the desired peptide chains. The final cleavage of side-chain protecting groups and the release of the C-terminal amide moiety was achieved by the treatment with trifluoroacetic acid in the presence of scavengers. Peptides were purified by preparative highperformance liquid chromatography (>95% purity), and characterized by amino acid analysis and mass spectroscopy. Specific details regarding the synthesis of the present peptides are provided below.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “about” means plus or minus 10%.

As used herein, “optional” or “optionally” means that the subsequently described event or circumstance may or may not occur, and that the description includes instances where said event or circumstance occurs and instances where it does not.

As used herein, the terms “individual”, “patient”, or “subject” can be an individual organism, a vertebrate, a mammal (e.g., a bovine, a canine, a feline, or an equine), or a human. In a preferred embodiment, the individual, patient, or subject is a human.

As used herein, the phrases “therapeutically effective amount” and “therapeutic level” mean a peptide dose or plasma concentration in a subject, respectively, that provides the specific pharmacological effect for which the peptide is administered in a subject in need of such treatment, i.e., to reduce, ameliorate, or eliminate the effects or symptoms of renal disease. It is emphasized that a therapeutically effective amount or therapeutic level of a drug will not always be effective in treating the conditions/diseases described herein, even though such dosage is deemed to be a therapeutically effective amount by those of skill in the art. The therapeutically effective amount may vary based on the route of administration and dosage form, the age and weight of the subject, and/or the subject's condition, including the type and stage of the amyloidosis at the time that treatment commences, among other factors.

The terms “treatment” or “treating” as used herein with reference to renal diseases, refer to reducing, ameliorating or eliminating one or more symptoms or effects of the disease or condition.

A “therapeutic response” means an improvement in at least one measure of renal disease.

As used herein, the term “pharmaceutically-acceptable carrier” means a material for admixture with a pharmaceutical compound (e.g., a chimeric peptide) for administration to a patient as described, for example, in “Ansel's Pharmaceutical Dosage Forms and Delivery Systems”, Tenth Edition (2014).

### Abbreviations

The following abbreviations are used herein: “Ac”—acetyl, —NH₂-amide, AKI—acute kidney injury, AP—acute pancreatitis, PMCA4b—plasma membrane ATPase 4b; RNLS—renalase.

Amino acids are represented by the IUPAC abbreviations, as follows: Alanine (Ala; A), Arginine (Arg; R), Asparagine (Asn; N), Aspartic acid (Asp; D), Cysteine (Cys; C), Glutamine (Gln; Q), Glutamic acid (Glu; E), Glycine (Gly; G), Histidine (His; H), Isoleucine (Ile; I), Leucine (Leu; L), Lysine (Lys; K), Methionine (Met; M), Phenylalanine (Phe; F), Proline (Pro; P), Serine (Ser; S), Threonine (Thr; T), Tryptophan (Trp; W), Tyrosine (Tyr; Y), Valine (Val; V), Norleucine (Nle), and 2-aminobutyric acid (Aib).

The expression “[X²²⁰]—Ac-Renalase A (205-240)-NH₂” means a peptide comprising the amino acids of Renalase A at positions 205-240 with an amino acid “X” at position 220, an acyl group at the NH₂ terminus and an amido group at the COOH terminus of the peptide. Thus, the expression represents the peptide: Ac-Lys²⁰⁵-Ile²⁰⁶-Asp²⁰⁷-Val²⁰⁸-Pro²⁰⁹-Trp²¹⁰-Ala²¹¹-Gly²¹²-Gln²¹³-Tyr²¹⁴-Ile²¹⁵-Thr²¹⁶-Ser²¹⁷-Asn²¹⁸-Pro²¹⁹-X²²⁰-Ile²²¹-Arg²²²-Phe²²³-Val²³⁴-Ser²²⁵-Ile²²⁶-Asp²²⁷-Asn²²⁸-Lys²²⁹-Lys²³⁰-Arg²³¹-Asn²³²-Ile²³³-Glu²³⁴-Ser²³⁵-Ser²³⁶-Glu²³⁷-Ile²³⁸-Gly²³⁹-Pro²⁴⁰-NH₂. (SEQ ID NO:06). When X is Ala, this compound is sometimes referred to herein as “peptide 10” or “BP-1002”.

Similar expressions for other peptides of the invention have the corresponding meanings.

### Pharmaceutical Formulations

Pharmaceutical compositions suitable for use in the methods described herein can include one or more of the disclosed peptides and a pharmaceutically acceptable carrier or diluent.

The composition may be formulated for intravenous, subcutaneous, intraperitoneal, intramuscular, topical, oral, buckle, nasal, pulmonary or inhalation, ocular, vaginal, or rectal administration. In some embodiments, the peptides are formulated for intravenous, subcutaneous, intraperitoneal, intramuscular administration, or targeted tissue delivery such as in a solution, suspension, emulsion, liposome formulation, etc. The pharmaceutical composition can be formulated to be an immediate-release composition, sustained-release composition, delayed-release composition, etc., using techniques known in the art.

Pharmacologically acceptable carriers for various dosage forms are known in the art. For example, excipients, lubricants, binders, and disintegrants for solid preparations are known; solvents, solubilizing agents, suspending agents, isotonicity agents, buffers, and soothing agents for liquid preparations are known. In some embodiments, the pharmaceutical compositions include one or more additional components, such as one or more preservatives, antioxidants, stabilizing agents and the like.

Additionally, the disclosed pharmaceutical compositions can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In some embodiment, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Pharmaceutical compositions of the disclosure can be administered in combination with other therapeutics that are part of the current standard of care for the kidney disease, pancreatic disease or tissue injury, particularly those that may benefit from regulation of excessive immunoinflammation. For example, fenoldopam, discovered as a selective dopamine 1 receptor agonists, has been utilized for acute kidney injury and could be used with the subject peptides in treatment.

### Methods of Treatment

In the present invention, at least one peptide is administered to a patient (e.g., a human patient) suffering from kidney disease, including AKI and AP. In some embodiments, the therapeutically effective amount of the peptide is administered together with a pharmaceutically acceptable carrier. Suitable pharmaceutically acceptable carriers are well-known in the art, as discussed infra. A typical route of administration is parenterally (e.g., intravenously, subcutaneously, or intramuscularly), as is well understood by those skilled in the medical arts. Other routes of administration are, of course, possible. Administration may be by single or multiple doses. The amount of peptide administered and the frequency of dosing may be optimized by the physician for the particular patient.

SARS-CoV-2 viral infections cause morbidity and mortality and exhibit select patterns of tissue injury. Acute inflammatory injuries occurring in specific tissues can disseminate throughout the body and result in multi-organ failure and death. For example, a key feature of severe acute pancreatitis is the development of multi-organ injury with lung and renal leading the list of secondarily affected organs. Renal injury also occurs in less severe bouts of acute pancreatitis but rapidly reverses in this setting. Relevant to many types of acute injuries and this proposal is that damage in one organ can lead to dysfunction of another. A significant subset of patients with COVID-19, especially those with severe disease, are expected to exhibit distinct patterns of tissue injury. SARS-CoV-2 infection can injure the kidney; its most prominent effects are on the proximal renal tubule, the site of renalase (RNLS) synthesis. SARS-CoV-2 infection of the kidneys is expected to lower circulating RNLS levels, which will sensitize target tissues to injury by the virus and other factors. We anticipate that the kidneys and the pancreas will be key pathologic targets; lower plasma RNLS levels will increase renal injury and also sensitize the pancreas to AP. Injury of these organs will further reduce plasma RNLS levels and drive a negative feedback loop

Clinical studies indicate that both kidney injury and acute pancreatitis occur frequently in SARS-CoV-2 infections. Though clinical studies of the natural history of SARS-CoV-2 infections are just appearing, distinct patterns of injury are emerging. In one limited study of 55 patients, evidence of acute pancreatitis was observed in 17% and there was an 8% incidence of renal dysfunction. This hospitalized cohort appeared to largely have moderate and not severe disease. Another study reported that blood creatinine levels in COVID-19 patients corresponded to severity and the incidence of renal injury in COVID-19 patients has been reported in up to 1/3 of hospitalized patients. In 52 patients with severe SARS-CoV-2 infections, 17% required dialysis. The presence of underlying kidney disease, a condition associated with decreased plasma RNLS levels, greatly increases the risk of death from SARS-CoV-2. Injury by SARS-COV-2 to proximal renal tubule cells, the site of RNLS production, was reported in a pathology study.

Inflammatory responses to SARS-COV-2 infections: Based on clinical findings, SARS-COV-2 illness has been divided into the following stages: I) Initial viral response II) Pulmonary phase and III) Hyperinflammation. One study that documented the time course of these responses in patients with mild vs severe SARS-COV-2 illness found prominently elevated serum IL6 levels throughout a two-week course only in those with severe disease. It is relevant that elevated plasma IL6 is a marker of AP severity and can also injury kidneys. IL6 and its precursor IL1, are being examined in therapeutic trials for SARS-COV-2 infections. Another observation is that interferon-gamma (IFN-gamma) levels, thought to be important for suppressing viral infections, was suppressed in both mild and severe disease.

Renalase functions as a pro-survival factor in models of AKI and AP. RNLS is a 37 kD secretory protein that is primarily made by kidney proximal renal tubules cells but also produced in other tissues. Its major cellular target is a widely distributed plasma membrane calcium exporter, plasma membrane calcium ATPase 4b(PMCA4b). Activation of PMCA4b is required for RNLS's protective function in a cellular AP model and other tissues. Through the use of selective PMCA4b inhibitors and genetic deletion models, we have found that PMCA4b is required for RNLS to have protective cellular effects.

Provided herein are methods of treating kidney disease, AKI and AP, including kidney disease related to SARS-CoV-2 in a patient (e.g., a human patient) in need of such treatment which comprises administering to the patient one or more of the disclosed peptides together with a pharmaceutically acceptable carrier, in an amount effective to treat the disease.

### Therapeutically Effective Doses and Dosing Regimens

In some embodiment, the therapeutically effective dose of the peptide may be administered no more than once, twice, three, or four times within a three-month period.

Therapeutically effective doses and dosing regimens of the foregoing methods may vary, as would be readily understood by those of skill in the art. Dosage regimens may be adjusted to provide the optimum desired response. For example, in some embodiments, a single dose of the peptide may be administered, while in some embodiments, several divided doses may be administered over time, or the dose may be proportionally reduced or increased in subsequent dosing as indicated by the situation. For example, in some embodiments the disclosed peptides may be administered once or twice weekly by subcutaneous, intravenous, or intramuscular injection. In some embodiments, the disclosed peptides may be administered once or twice monthly by subcutaneous, intravenous, or intramuscular injection. In some embodiments, the disclosed peptides thereof may be administered once or twice annually by subcutaneous, intravenous, or intramuscular injection. In some embodiments, the disclosed peptides may be administered once every week, once every other week, once every three weeks, once every four weeks, once every month, once every other month, once every three months, once every four months, once every five months, once every six months, once every seven months, once every eight months, once every nine months, once every ten months, once every eleven months, twice a year, or once a year, as the situation or condition of the patient may indicate.

The therapeutically effective dose of peptide administered to the patient (whether administered in a single does or multiple doses) should be sufficient to treat renal disease or AP. Such therapeutically effective amount may be determined by evaluating the symptomatic changes in the patient.

Exemplary doses can vary according to the size and health of the individual being treated, as well as the condition being treated. In some embodiments, the effective amount of a disclosed peptide is about 2,200 mg; however, in some situations the dose may be higher or lower. In some embodiments, a therapeutically effective amount may be between 50 and 5000 mg, between 60 about 4500 mg, between 70 and 4000 mg, between 80 and 3500 mg, between 90 and 3000 mg, between 100 and 2500 mg, between 150 and 2000 mg, between 200 and 1500 mg, between 250 and 1000 mg, or any dose in between. For instance, in some embodiments, the therapeutically effective amount may be about 50 about 60, about 70, about 80, about 90, about 100, about 150, about 200, about 250, about 300, about 350, about 400, about 450, about 500, about 550, about 600, about 650, about 700, about 750, about 800, about 850, about 900, about 950, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, about 3000, about 3100, about 3200, about 3300, about 3400, about 3500, about 3600, about 3700, about 3800, about 3900, about 4000, about 4100, about 4200, about 4300, about 4400, about 4500, about 4600, about 4700, about 4800, about 4900, about 5000 or more mg.

Similarly, in some embodiments, the effective amount of peptide is about 25 mg/kg; however, in some embodiments, the concentration may be higher or lower. In some embodiments, the effective amount may be about 1-50 mg/kg, about 5-40 mg/kg, about 10-30 mg/kg, or about 15-25 mg/kg or any value in between. For instance, in some embodiments, the effective amount may be 1, 2, 3, 4, 5, 6, 7, 8, 9, 10, 11, 12, 13, 14, 15, 16, 17, 18, 19, 20, 21, 22, 23, 24, 25, 26, 26, 28, 29, 30, 31, 32, 33, 34, 35, 36, 37, 38, 39, 40, 41, 42, 43, 44, 45, 46, 47, 48, 49, or 50 or more mg/kg.

The disclosed methods of treatment may also be combined with other known methods of treatment as the situation may require. For example, renalase agonists may offset the potential ^{.}cytokine storm' that leads to excessive tissue damage and death in SARS-CoV-2 and potentially other respiratory viral disease. For example, the H1 N1 pandemic resulted in extensive kidney damage. The examples below reflect SARS-CoV-2 from current literature.

### Potential Drug Combinations

| Drug/diagnostic | | | |
|---|---|---|---|
| Potential | Rationale | Comment/ref | Comb? |
| Alpha adrenergic | Alpha receptors and | Preventing ‘Cytokine Storm’ May | Yes |
| receptor blockers | catecholamines | Ease Severe COVID-19 Symptoms | |
| (example Tamulosin) | may be important step | https://www.hhmi.org/news/ | |
| or block of | in cytokine storm. | preventing-cytokine-storm-may-ease- | |
| catecholamine synthesis | Blockers may be | severe-covid-19-symptoms; | |
| (alpha-methyl tyrosine) | therapeutic | https://www.nature.com/articles/ | |
| | | s41586-018-0774-y | |
| IL-6 receptor blockade; | IL-6, pro-inflammatory | https://www.idse.net/Covid-19/Article/ | Yes. |
| tocilizumab (Actemra, | cytokine elevations | 03-20/COVID-19-Brings-Cytokine-Storm/58061 | Potentially |
| Genentech) | in covid patients; | https://www.accessmedicinenetwork.com/ | important |
| | block IL-6 Receptors | channels/2610-accessmedicine-covid-19- | combination |
| | | central/posts/62262-treatment | |
| IL-1 blocker, anakinra | | https://www.idse.net/Covid-19/Article/ | Yes |
| (Kineret, Amgen) | | 03-20/COVID-19-Brings-Cytokine-Storm/58061 | |
| Diagnostic test | Example-our Elisa | Potential theranostic for RNLS agonist to | Yes |
| of decreased renalase | test for RNLS | patients with reduced RNLSin serum or plasma | |
| Diagnostic test | | RNLS agonist theranostic with 1 | Yes |
| for renalase damage | | or more of these tests | |
| biomarkers; elevated | | | |
| creatinine; >BUN; >NGAL | | | |
| Elevated C-reactive | Covid biomarkers | Following elevated levels of the | Yes |
| protein and D-dimer | | markers of covid damage or inflammation | |
| levels | | https://www.ncbi.nlm.nih.gov/pmc/ | |
| | | articles/PMC7141628/ | |
| | | https://www.aacc.org/publications/ | |
| | | cln/cln-stat/2020/april/16/key- | |
| | | biomarkers-in-managing-covid-19 | |
| serum ferritin | Covid biomarker of | https://www.idse.net/Covid-19/Article/ | Possible |
| levels elevated | pathophysiology | 03-20/COVID-19-Brings-Cytokine-Storm/58061 | utility |
| | | https://www.drugtargetreview.com/news/60769/ | |
| | | hyperferritinaemia-could-be-causing-severe- | |
| | | covid-19/ | |
| Passive immunity | | Potential utility for combination. | Possible |
| With antibodies | | Perhaps slow rejection with RNLS agonists; | utility |
| from recovered | | This is speculative | |
| Covid patients | | | |
| Reduced platelet | Marker of Covid | https://www.aacc.org/publications/ | Possible |
| levels diagnosis | severity | cln/cln-stat/2020/april/16/key- | utility |
| | | biomarkers-in-managing-covid-19 | |

Note:
“Comb?” represents possible combination of noted therapy with RNLS or a RNLS agonist

Therapeutically effective doses and dosing regimens of the foregoing methods may vary, as would be readily understood by those of skill in the art. Dosage regimens may be adjusted to provide the optimum desired response. For example, in some embodiments, a single bolus dose of the peptide may be administered, while in some embodiments, several divided doses may be administered over time, or the dose may be proportionally reduced or increased in subsequent dosing as indicated by the situation. For example, in some embodiments the disclosed peptides may be administered once or twice weekly by subcutaneous, intravenous, or intramuscular injection. In some embodiments, the disclosed peptides may be administered once or twice monthly by subcutaneous, intravenous, or intramuscular injection. In some embodiments, the disclosed peptides may be administered once or twice annually by subcutaneous, intravenous, or intramuscular injection. In some embodiments, the disclosed peptides may be administered once every week, once every other week, once every three weeks, once every four weeks, once every month, once every other month, once every three months, once every four months, once every five months, once every six months, once every seven months, once every eight months, once every nine months, once every ten months, once every eleven months, twice a year, or once a year, as the situation or condition of the patient may indicate.

### Examples: Peptide Synthesis

The peptides were synthesized on a ChemMatrix Rink Amide resin, using standard Fmoc-synthesis protocol on an APEX 396 automatic synthesizer. For removal of the Fmoc-protecting group: The resin was swollen in N,N-dimethylformamide (DMF) for 30 minutes, treated at 50° C. with 20% piperidine-DMF for 8 minutes, and washed three times with DMF. For the coupling reaction: The Fmoc-protected amino acid, 6-chloro-1-hydroxybenzotriazole (Cl-HOBt), diisodopropyl-carbodiimide (DCI) and N-methyl-2-pyrrolidine (NMP) were added to the resin. The mixture was vortexed for 20 minutes at 50° C. Afterwards, the resin was washed with DMF once. The cycle of Fmoc-deprotection and coupling steps was repeated until the last amino acid residue was assembled. After removal of the final Fmoc-protecting group, the resin was treated with 20% acetic anhydride-NMP for 20 minutes and was then washed with DMF, methylene chloride (DCM), and dried with air. The peptides were cleaved from the resin using a trifluoroacetic acid (TFA) cocktail [95% TFA, 2.5% water, and 2.5% triisopropylsilane (TIS)) for three hours. Crude peptides were precipitated by adding ice-chilled anhydrous ethyl ether, washed three times with anhydrous ethyl ether, and dried in vacuo. Some examples of specific representative syntheses are given below.

[Ala²²⁰]-Ac-Renalase A (205-240)-CONH₂ (peptide 10): Synthesized by solid phase synthesis as outlined above. It was determined to be >95% pure by high performance liquid chromatography (HPLC) and confirmed by mass spectroscopy and amino acid analysis.

[Ser²²⁰]-Ac-Renalase A (205-240)-CONH₂: Synthesized by solid phase synthesis as outlined above. It was determined to be >95% pure by high performance liquid chromatography (HPLC) and confirmed by mass spectroscopy and amino acid analysis.

[Ala²²⁰]-Ac-Renalase A (214-240)-CONH₂: Synthesized by solid phase synthesis as outlined above. It was determined to be >95% pure by high performance liquid chromatography (HPLC) and confirmed by mass spectroscopy and amino acid analysis.

Ac-Renalase A (205-240)-CONH₂: Synthesized by solid phase synthesis as outlined above. It was determined to be >95% pure by high performance liquid chromatography (HPLC) and confirmed by mass spectroscopy and amino acid analysis.

[Ala²²⁰]-Ac Renalase A (205-253)-CONH₂: Synthesized by solid phase synthesis as outlined above. It was determined to be >95% pure by high performance liquid chromatography (HPLC) and confirmed by mass spectroscopy and amino acid analysis.

Renalase A (205-240)-OH (peptide 81): Synthesized by solid phase synthesis as outlined above using 2-chlorotrityl-resin in place of the Rink-Amide resin. It was determined to be >95% pure by high performance liquid chromatography (HPLC) and confirmed by mass spectroscopy and amino acid analysis.

### Examples: Testing

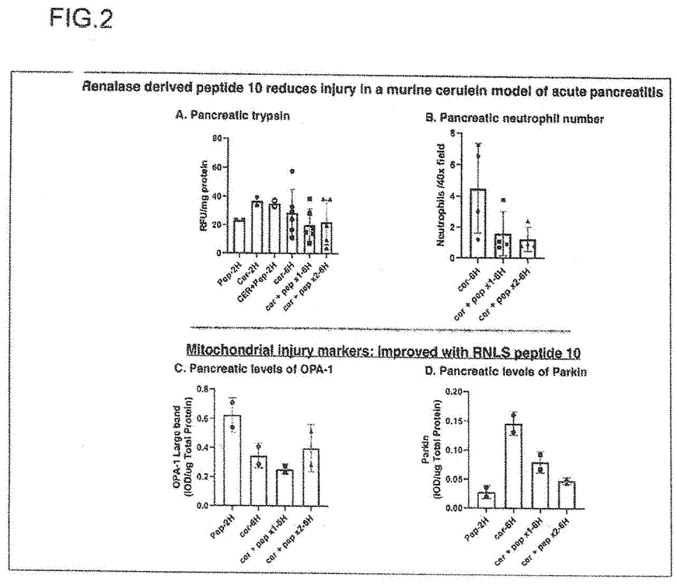

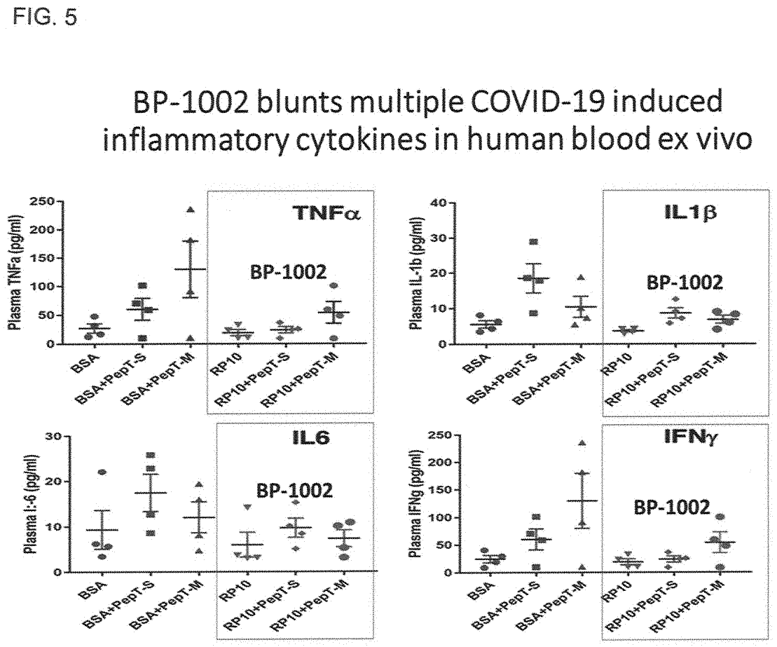

Renalase Agonists reduce injury in experimental AP: In vivo studies using the RNLS agonist [Ala²²⁰]-Ac-Renalase A (205-240)-NH₂ (peptide 10) show promising and dramatic effects on AP responses (FIG. 2) Though we found no effects on the earliest responses (not shown), which are acinar cell related, the later responses (6 hrs) that are mediated largely by inflammatory cells showed that peptide 10 tended to reduce levels of active trypsin (2A), dramatically decrease tissue neutrophils (2B), and reduced mitochondrial injury as determined by OPA-1 and Parkin levels (2C, D). This suggests that peptide 10 has a much greater effect on reducing pancreatitis injury for events after the first 1-2 hrs of AP. This likely represents inhibition of inflammatory pathways central to AP pathogenesis; a probable target is inflammatory activation (FIG. 5). Since most patients with AP present long after disease onset and later onset responses are most important in determining AP severity, the beneficial effects of peptide 10 should be therapeutically valuable even late in the course of disease. In this context we have shown that RNLS agonists reduce injury when given 2 hrs after the onset of cerulein AP and 12hrs after the induction of arginine AP.

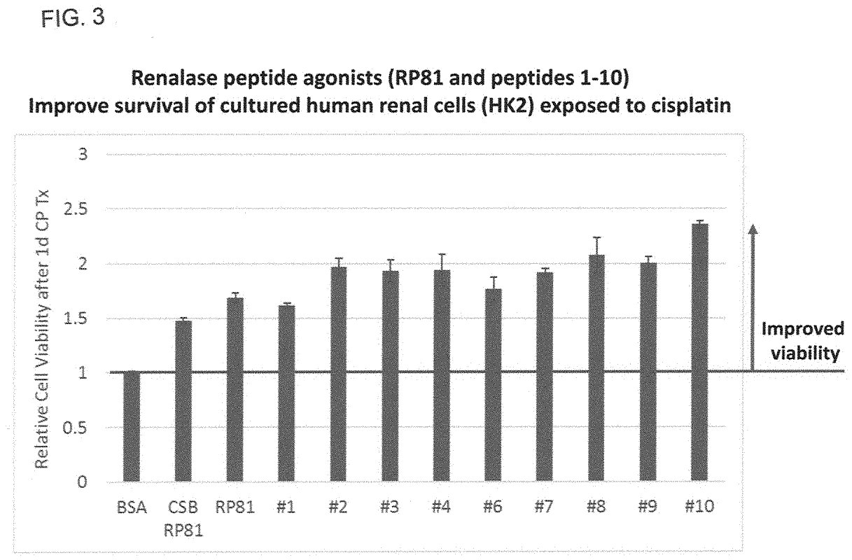

RNLS peptide agonists reduce injury in renal models: We have found that RNLS peptide agonists are also protective in models of renal injury. We have shown that a peptide of the invention [Ala²²⁰]-Ac-Renalase A (205-240)-NH₂ eliminated cisplatin-induced injury of cultured kidney cells. In FIG. 3 we show that the peptide reduces injury in a cultured proximal renal tubule cell line. We also showed that this RNLS agonist peptide reduced renal ischemia reperfusion injury in a preclinical murine model (FIG. 3). Data are shown below for such RNLS agonists

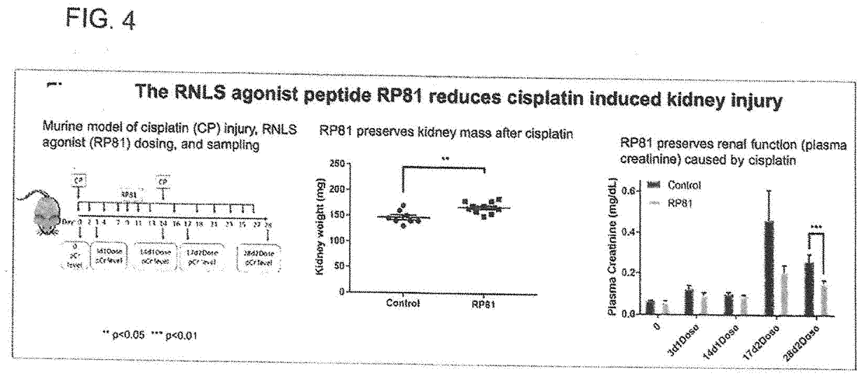

FIG. 4 shows that an RNLS agonist Renalase A (205-240)-OH (peptide 81) reduces kidney injury after cisplatin-induced treatment. A key measure of this toxicity is a decrease in kidney mass; this is limited when Renalase A (205-240)-OH is given. More dramatic is the effect of peptide on the reduced renal function seen with cisplatin induced renal injury. As seen in the right panel of FIG. 4, plasma creatinine levels increase 4-fold after 17 days of cisplatin treatment; the RNLS agonist reduced this toward the normal range. This preliminary data suggest that RNLS agonists can reduce injury to renal cells and also is effective in vivo AKI models.

The RNLS agonist [Ala²²⁰]-Ac-Renalase A (205-240)-NH₂ reduces COVID19-induced innate immune responses (FIG. 5): We have found that RNLS agonists can have anti-inflammatory effects. Amplified inflammatory responses mediate severe pancreatitis, renal damage, and SARS-CoV-2 infections. With SARS-CoV-2, these could be elicited by responses to viral antigens. To address this issue, we exposed blood from healthy donors to mixtures of peptides contained in three major SARS-CoV-2 capsid proteins (SARS-CoV-2 proteins S, M or N) from Miltenyi Biotec ata concentration of 0.19 nM in blood samples. To this, a buffer mock control or the RNLS peptide was added at 50 ug/ml. After 3 hrs of incubation samples were assayed for cytokine production by ELISA and compared by 2way ANOVA/Turkey's comparison. As shown in FIG. 5, the SARS-CoV-2 peptides stimulated cytokine responses to different extents; though Peptide S and M gave robust responses for each response (P<0.01), there was little response Peptide N (not shown). SARS-CoV-2 peptides S and M both increased the levels of interferon-gamma; this response was dramatically decreased by the RNLS agonist [Ala²²⁰]-Ac-Renalase A (205-240)-NH₂. Since interferon-gamma is thought to be important for inhibited viral growth, this RNLS agonist effect needs to be avoided and is discussed below. However, other potentially beneficial effects were observed. Thus, [Ala²²⁰]-Ac-Renalase A (205-240)-NH₂, peptide 10, inhibited the SARS-CoV-2-peptide induced increases TNF-alpha, and IL1-beta and IL6 (p<0.01). These innate immune responses have been implicated in causing both renal and pancreatic damage. They are also judged central to the hyper-inflammatory state that characterizes severely ill SARS-CoV-2 infections and causes many deaths.

The RNLS agonist [Ala²²⁰]-Ac-Renalase A (205-240)-NH₂ exhibits anti-inflammatory activity as determined by carrageenan-induced Paw edema.

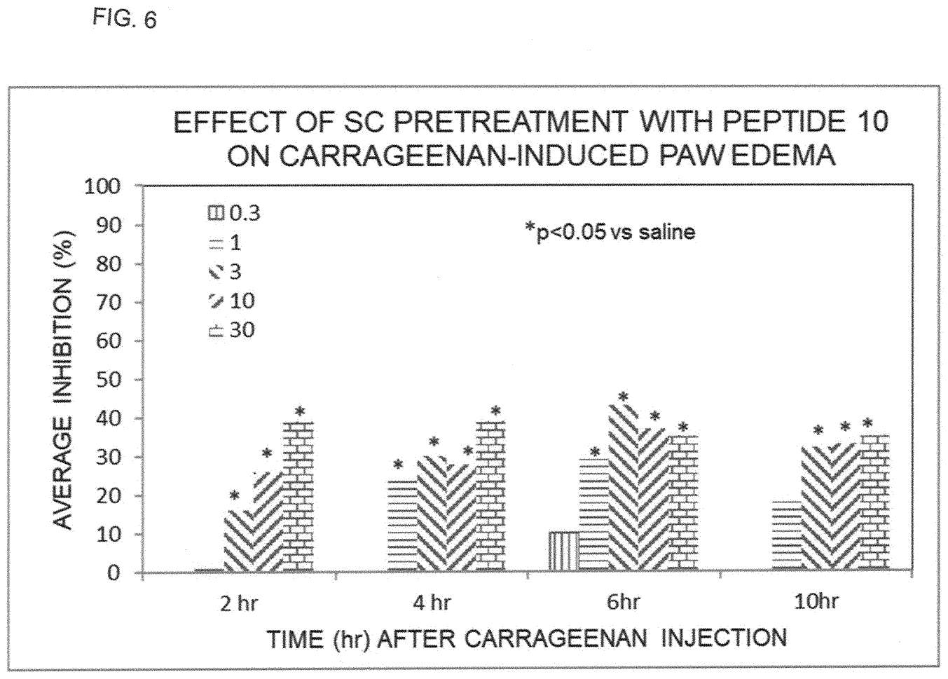

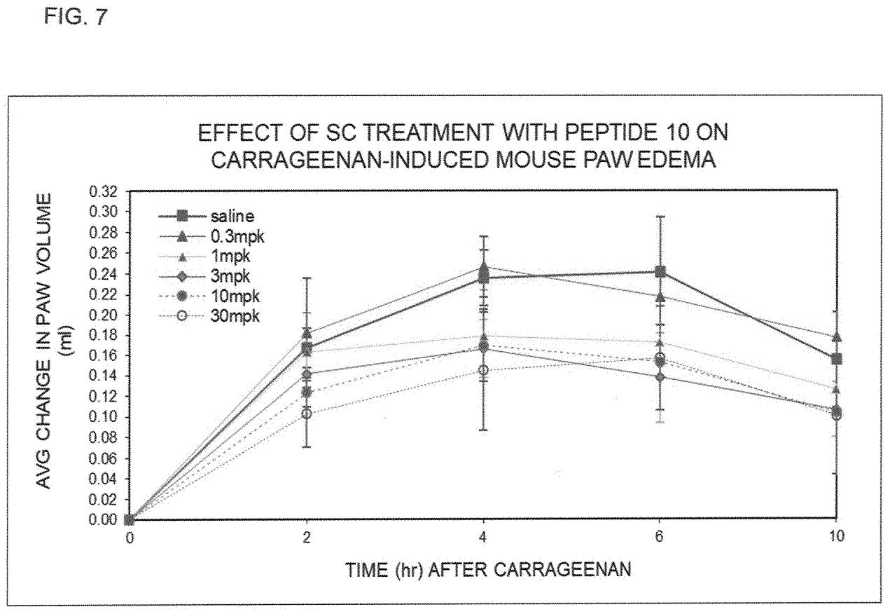

This test is a rapid in vivo model for evaluation of anti-inflammatory efficacy of test articles. The CPE model in mice quantitativdy assesses inhibition of edema enduced by subplantarinjection of carrageenan. Standard test period is 6 hours with edema measured at 0, 2, 4 and 6 hours—the results here show a test period of 10 hours. Carrageenan-induced inflammation invoves extravasation of polymorphonuclear leukocytes from circulation to the site of inflammation. The subsequent release of myeloperoxidase and other cytokines within the interstitial tissues results in plasma exudation into the site of inflammation. The increased paw volume is measured by water displacement. Anti-inflammatory drugs reduce the paw edema. Results shown in FIGS. 6 and 7 (same data in two different formats) demonstate that peptide 10, [Ala²²⁰]-Ac-Renalase A(205-240)-NH₂, has an anti-inflammatory effect in the model.

### Procedures

1. Receive (SOP 1910, SOP 1920) and quarantine (SOP 560) 125 CD-1 mice (Charles River Laboratories, male, 5-6 weeks old).
2. Animals are ear tagged (SOP 810), weighed, and sorted into 9 groups with 10 animals/group and 5 groups of 7 animals/group based upon average body weight.
3. Prepare 3% carrageenan solution:
   1) Preparation at least 2 weeks in advance of use.
   2) Weigh out 600 mg A-carrageenan into a glass beaker.
   3) Add 20 ml deionized water.
   4) Stir on mild heat until carrageenan fully dissolved.
   5) Cool solution to ambient temperature.
   6) Store at 4-8° C.
4. Until the day of use the renalase peptides are stored at -20° C. as dry powder.
5. DAY 0
   a. Prepare test material in sterile saline (vehicle).
   b. On the day of dosing, renalase peptides are dissolved.
   c. On day prior to administration check carrageenan solution for clarity (no visible evidence of contamination).
      1) Equilibrate carrageenan to ambient temperature.
   d. Record mouse weights in laboratory notebook.
   e. Measure and record paw initial paw volumes:
      1) Place beaker of water on balance and tare to zero.
      2) Place right hind limb in the beaker of water on the scale with the top of the hock centered in the meniscus of the water. Record read-out on the scale.
      3) The foot must be dried after each measurement as wet feet will change the readout.
      4) Re-tare the scale between measurements to account for decrease in water volume.
   f. Vehicle and test materials are administered at 10 mg/kg by subcutaneous (SC, SOP 1610) injection at 30 min prior to administration of carrageenan as in TABLE 1.

TABLE 1
GROUP TREATMENTS

| Group | No. Mice | Treatment | Dose (mg/kg) | ROA | Regimen |
|---|---|---|---|---|---|
| 1 | 10 | Saline | 10 mg/kg | SC | −0.5 hr |
| 2 | ″ | Peptide 10 | 0.3 | ″ | ″ |
| 3 | ″ | ″ | 1 | ″ | ″ |
| 4 | ″ | ″ | 3 | ″ | ″ |
| 5 | ″ | ″ | 10 | ″ | ″ |
| 6 | ″ | ″ | 30 | ″ | ″ |

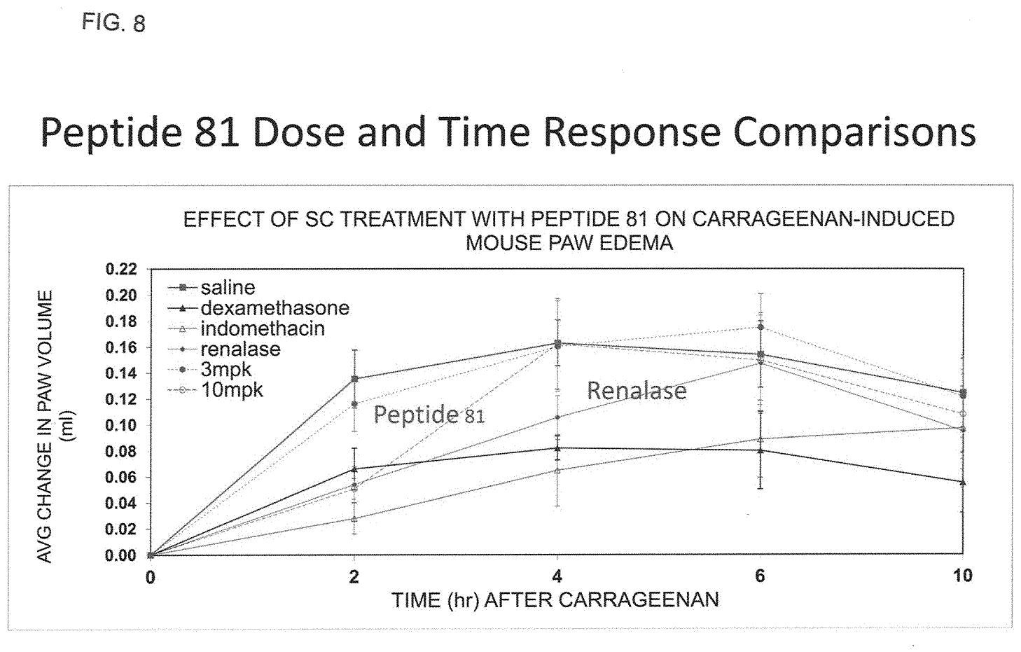

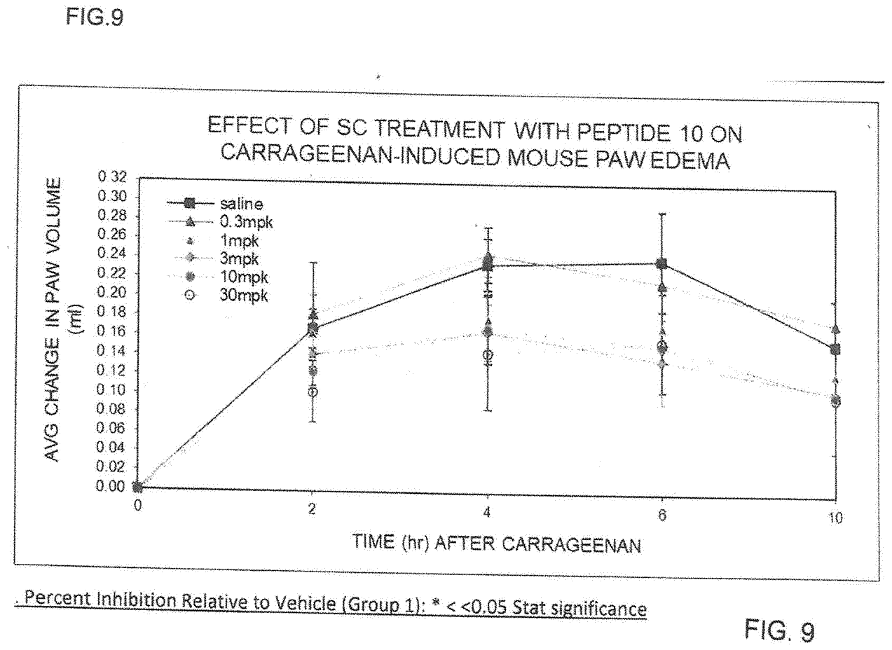

The test was also conducted to compare the inhibition of inflammation by peptide 10 as compared to the corresponding peptide fragment without the alanine substitution at position 220 (peptide 81). Results are shown in FIGS. 8 and 9 and in Table 2 below, where the figures represent percent change from control and * means <<0.05 statistical significance.

TABLE 2
RESULTS

| | | Time After Carrageenan | | | |
|---|---|---|---|---|---|
| Group | Treatment | 2 hr | 4 hr | 6 hr | 10 hr |
| 2 | Dexamethasone, 10 mpk | 51.2* | 49.7* | 48.1* | 55.6* |
| 3 | Indomethacin, 5 mpk | 79.4* | 60.3* | 42.4* | 21.3 |
| 4 | Renalase, 3 mpk | 60.1* | 35.3* | 4.5 | 23.7 |
| 5 | Peptide 81, 3 mpk | 14.2 | 1.6 | −13.1 | 2.4 |
| 6 | Peptide 81, 10 mpk | 62.6* | 0.5 | 2.9 | 13.3 |

The results indicate peptide 10, [Ala²²⁰]-Ac-Renalase A (205-240)-NH₂, was more active and longer active than peptide 81, Ac-Renalase A (205-240)-OH, consistent with peptide 10 being more stable than peptide 81. Note peptide 10 had activity more in line with standards dexamethasone and indomethacin. Results were from comparable but different experiments done by same group with same protocols but drugs differed as shown. Note for example at 2 hours both peptide 10 and peptide 81 were active but only peptide 10 was active for longer periods of time.

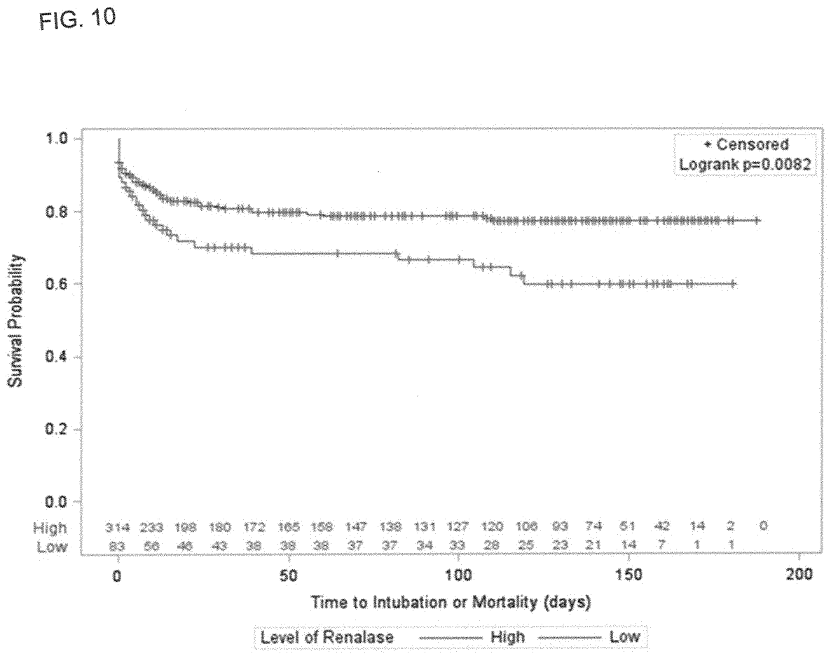

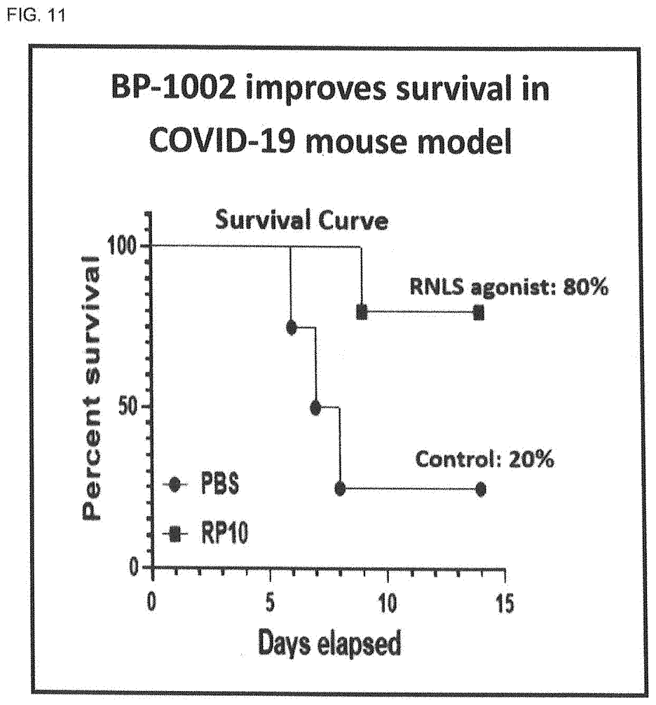

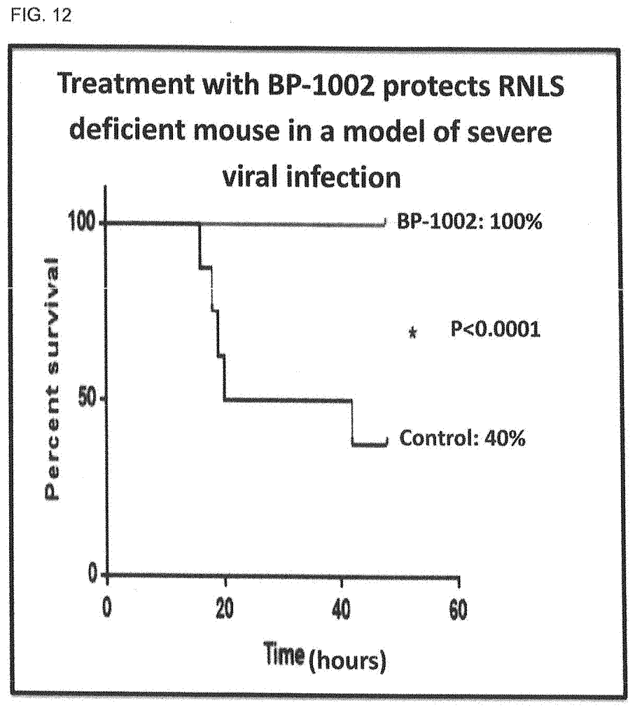

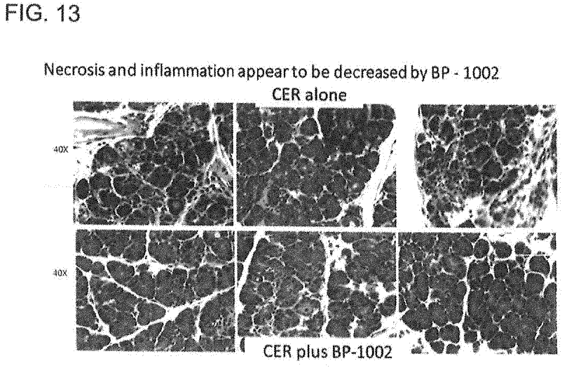

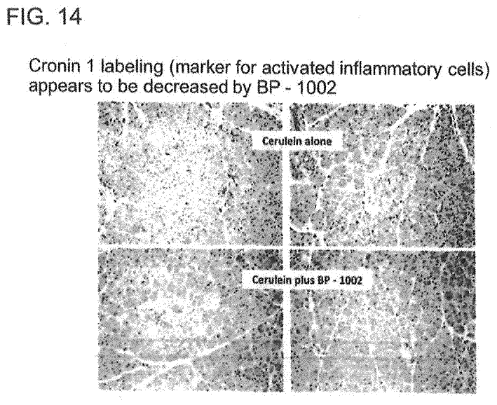

Additionally, decreased renalase activity is correlated with increased mortality in COVID-19 as shown in FIG. 10, while treatment with renalase agonist BP-1002 has beneficial effects in COVID-19, other infections, and inflammation, as shown in FIG. 11-14.

All references cited herein are incorporated herein by reference as if included in their entirety.

In the description and claims of this specification the word “comprise” and variations of that word, such as “comprises” and “comprising” are not intended to exclude other features, additives, components, integers or steps but rather, unless otherwise stated explicitly, the scope of these words should be construed broadly such that they have an inclusive meaning rather than an exclusive one.

Although the compositions and methods of the invention have been described in the present disclosure by way of illustrative examples, it is to be understood that the invention is not limited thereto and that variations can be made as known by those skilled in the art without departing from the teachings of the invention defined by the appended claims.

What is claimed is:

1. A peptide comprising
R-X²²⁰-Ile²²¹-Arg²²²-Phe²²³-Val²³⁴-Ser²²⁵-Ile²²⁶-Asp²²⁷-Asn²²⁸-Lys²²⁹-R′
wherein the superscripts represent positions within the renalase A chain (1-342), R is selected from Ac-Ala-Gly-Thr-, Ac-Gly-Thr-, Ac-Thr—, Ac- , Ac-Z, H-, H-Z, B-Z-, and B, wherein Z is selected from one or more of the amino acid residues at positions 205 — 219 of the renalase A chain, R′ is selected from —NH₂, Z′—NH₂, —B, and Z′—B, wherein Z′ is selected from one or more of the amino acid residues at positions 230-253 of the renalase A chain, B is PEG or bis-PEG, and X is the residue of an amino acid selected from: 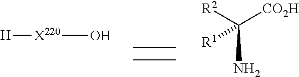
wherein R^{l} and R² are independently H; C₁ to C₈ n-alkyl optionally substituted by hydroxyl; C3 to C8 branched alkyl optionally substituted by hydroxyl; C₄ to C₈ doubly branched alkyl optionally substituted by hydroxyl; C₃ to C₆ cycloalkyl optionally substituted by hydroxyl or methyl or both at any position or positions and which includes all structurally feasible stereoisomeric entities; CH₂—C₃ to C₆ cycloalkyl optionally substituted by hydroxyl or methyl or both at any position or positions and R^{l} and R² may be linked together as (CH₂)ₙ optionally substituted at any position by methyl or hydroxyl or both, wherein n is 2, 3, 4, or 5; and 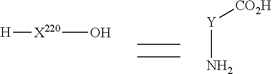
wherein Y is (CH₂)ₙ optionally substituted at any position or positions by methyl or hydroxyl or both, provided that the carbon bearing the amino group may be substituted only by methyl, wherein n is 2, 3, 4, 5; cis- or trans-1,2-cyclopropanediyl, cis- or trans-1,2-cyclobutanediyl, cis- or trans-1,3 -cyclobutanediyl, cis- or trans-1,2-cyclopentanediyl, cis- or trans- 1,3 -cyclopentanediyl, cis- or trans-1,2-cyclohexanediyl, cis- or trans-1,3-cyclohexanediyl, or cis- or trans-1,4-cyclohexanediyl optionally substituted by at any position or positions by methyl or hydroxyl or both provided that the carbon bearing the amino group may be substituted only by methyl and which includes all structurally feasible diastereoisomeric entities.

2. The peptide of claim 1 that is:
R- Lys²²⁰-Ile²⁰⁶-Asp²⁰⁷-Val²⁰⁸-Pro²⁰⁹-Trp²¹⁰-Ala¹¹-Gly²¹²-Gln²¹³-Tyr²¹⁴-Ile²¹⁵-Thr²¹⁶Ser²¹⁷-Asn²¹⁸-Pro²¹⁹-X²²⁰-Ile²²¹-Arg²²²-Phe²²³-Val²³⁴-Ser²²⁵-Ile²²⁶-Asp²²⁷-Ash²²⁸-Lys²²⁹-Lys²³⁰-Arg²³¹-Ash²³²-Asn²³²-Ile²³³-Glu²³⁴-Ser²³⁵-Ser²³⁶-Glu²³⁷-Ile²³⁸-Gly²³⁹-Pro²⁴⁰-Ser²⁴¹-Leu²⁴²-Val²⁴³-Ile²⁴⁴-His²⁴⁵-Thr²⁴⁶-Thr²⁴⁷-Val²⁴⁸-Pro²⁴⁹-Phe²⁵⁰-Gly²⁵¹-Val²⁵²-Thr²⁵³-R′ wherein R is selected from Ac-Ala-Gly-Thr-, Ac-Gly-Thr-, Ac-Thr—, Ac-, H-, PEG, and bis-PEG, R′ is selected from -Tyr-Leu-Glu-NH₂, Tyr-Leu-NH₂, -Tyr-NH₂, -NH₂, -OH, and PEG, and X²²⁰ is selected from Gly, Ser, Ala, Leu, Val, Ile, Nle, β-Ala cyclopropyl-Gyl, (cyclopropylmethyl)-Gly, and Aib.

3. The peptide of claim 2 wherein R is Ac-.

4. (canceled)

5. (canceled)

6. The peptide of claim 1 which is [Ala²²⁰]-Ac-Renalase A (205-253)-NH₂.

7. The peptide of claim 1 which is R- Lys²⁰⁵-Ile²⁰⁶-Asp²⁰⁷-Val²⁰⁸-Pro²⁰⁹-Trp²¹⁰-Ala²¹¹ -Gly²¹²-Gln²¹³-Tyr²¹⁴-Ile²¹⁵-Thr²¹⁶-Ser²¹⁷-Ash²¹⁸-Pro²¹⁹-X²²⁰-Ile²²¹-Arg²²²-Phe²²³-Val²³⁴-Ser²²⁵-Ile²²⁶-Asp²²⁷-Ash²²⁸-Lys²²⁹-Lys²³⁰-Arg²³¹-Ash²³²-Ile²³³-Glu²³⁴-Ser²³⁵-Ser²³⁶-Glu²³⁷-Ile²³⁸-Gly²³⁹-Pro²⁴⁰-R′.

8. The peptide of claim 7 wherein R represents Ac-Ala-Gly-Thr-, Ac-Gly-Thr-, Ac-Thr—, Ac-, or H-.

9. (canceled)

10. The peptide of claim 7 wherein R′ represents -Ser-Leu-Val-NH₂, -Ser-Leu-NH₂, -Ser-NH₂, —NH₂, or —OH

11. The peptide of claim 7 wherein R′ represents a linear chain of poly(ethylene)glycol [PEG] with an average molecular weight of between 500 and 20,000 amu.

12. The peptide of claim 7 which is [bis-PEG5000]-Lys²⁰⁵-Ile²⁰⁶-Asp²⁰⁷-Val²⁰⁸-Pro²⁰⁹-Trp²¹⁰-Ala²¹¹-Gly²¹²-Gln²¹³-Tyr²¹⁴-Ile²¹⁵-Thr²¹⁶-Ser²¹⁷-Asn²¹⁸-Pro²¹⁹-Ala²²⁰-Ile²²¹-Arg²²²-Phe²²³-Val²³⁴-Ser²²⁵-Ile²²⁶-Asp²²⁷-Asn²²⁸-Lys²²⁹-Lys²³⁰-Arg²³¹-Asn²³²-Ile²³³-Glu²³⁴-Ser²³⁵-Ser²³⁶-Glu²³⁷-Ile²³⁸-Gly²³⁹-Pro²⁴⁰-NH₂.

13. The peptide of claim 7 wherein X²²⁰ represents Gly, Ser, Ala, Leu, Val, Ile, Nle, β-Ala, cyclopropyl-Gyl, (cyclopropylmethyl)-Gly, or Aib.

14. The peptide according to claim 13 wherein R is Ac-.

15. (canceled)

16. (canceled)

17. The peptide according to claim 13 which is [Ala²²⁰]-Ac-Renalase A (205-240)-NH₂.

18. The peptide of claim 1 that is R-Tyr²¹⁴-Ile²¹⁵-Thr²¹⁶-Ser²¹⁷-Asn²¹⁸-Pro²¹⁹-X²²⁰-Ile²²¹-Arg²²²-Phe²²³-Val²³⁴-Ser²²⁵-Ile²²⁶-Asp²²⁷-Asn²²⁸-Lys²²⁹-Lys²³⁰-Arg²³¹-Asn²³²-Ile²³³-Glu²³⁴-Ser²³⁵-Ser²³⁶-Glu²³⁷-Ile ²³⁸-Gly²³⁹-Pro²⁴⁰-R′.

19. The peptide of the claim 18 wherein R represents Ac-Ala-Gly-Thr- or Ac-Gly-Thr- or Ac-Thr— or Ac- or H-.

20. (canceled)

21. The peptide of claim 18 wherein R′ represents -Ser-Leu-Val-NH₂ or -Ser-Leu-NH₂ or -Ser-NH₂ or —NH₂ or —OH

22. (canceled)

23. The peptide of claim 18 wherein X²²⁰ represents Gly, Ser, Ala, Leu, Val, Ile, Nle, β-Ala, cyclopropyl-Gyl, (cyclopropylmethyl)-Gly, or Aib.

24. The peptide according to claim 23 wherein R is Ac-.

25. (canceled)

26. (canceled)

27. The peptide according to claim 23 which is [Ala²²⁰]-Ac-Renalase A (214-240)-NH₂.

28. The peptide of claim 1 that is R-Tyr₂₁₄-Ile²¹⁵-Thr²¹⁶-Ser²¹⁷-Asn²¹⁸-Pro²¹⁹-X²²⁰-Ile²²¹-Arg²²²-Phe²²³-Val²³⁴-Ser²²⁵-Ile²²⁶-Asp²²⁷-Ash²²⁸-Lys²²⁹-Lys²³⁰-Arg²³¹-Asn²³²-Ile²³³-Glu²³⁴-Ser²³⁵-Ser²³⁶-Glu²³⁷-Ile²³⁸-Gly²³⁹-Pro²⁴⁰-Ser²⁴¹-Leu²⁴²-Val²⁴³-Ile²⁴⁴-His²⁴⁵-Thr²⁴⁶-Thr²⁴⁷-Val²⁴⁸-Pro²⁴⁹-Phe²⁵⁰-Gly²⁵¹-Val²⁵²-Thr²⁵³-R′.

29. The peptide of the claim 28 wherein R represents Ac-Ala-Gly-Thr- or Ac-Gly-Thr- or Ac-Thr— or Ac- or H-.

30. (canceled)

31. The peptide of claim 28 wherein R′ represents -Ser-Leu-Val-NH₂ or -Ser-Leu-NH₂ or -Ser-NH₂ or —NH₂ or —OH

32. (canceled)

33. The peptide of claim 28 wherein X²²⁰ represents Gly, Ser, Ala, Leu, Val, Ile, Nle, β-Ala, cyclopropyl-Gyl, (cyclopropylmethyl)-Gly, or Aib.

34. The peptide according to claim 33 wherein R is Ac-.

35. The peptide according to claim 34 wherein R′ is —NH₂

36. The peptide according to claim 35 wherein X²²⁰ is Ala.

37. The peptide according to claim 36 which is [Ala²²⁰]-Ac-Renalase A (214-253)-NH₂.

38. A method of treating acute kidney injury or acute pancreatitis in a patient in need of such treatment that comprises administering to the patient a therapeutically-effective amount of the peptide of claim 1.

39. A pharmaceutical composition comprising the peptide of claim 1 admixed with a pharmaceutically-acceptable carrier.


SEQUENCE LISTING (20 sequences)

SEQ ID NO: 1 - protein, 342 aa, Homo sapiens
MAQVLIVGAGMTGSLCAALLRRQTSGPLYLAVWDKADDSGGRMTTACSPHNPQCTADLGAQYITCTPHYAKKHQRFYDELLAYGVLRPLSSPIEGMVMKEGDCNFVAPQGISSIIKHYLKESGAEVYFRHRVTQINLRDDKWEVSKQTGSPEQFDLIVLTMPVPEILQLQGDITTLISECQRQQLEAVSYSSRYALGLFYEAGTKIDVPWAGQYITSNPCIRFVSIDNKKRNIESSEIGPSLVIHTTVPFGVTYLEHSIEDVQELVFQQLENILPGLPQPIATKCQKWRHSQVTNAAANCPGQMTLHHKPFLACGGDGFTQSNFDGCITSALCVLEALKNYI

SEQ ID NO: 2 - protein, 36 aa, Artificial Sequence
  Based on homo sapiens sequence.
  MISC_FEATURE (16)..(16): X is glycine, serine, alanina, leucine, valine, isoleucine, norleucine, cyclopropyl-glycine, (cyclopropylmethyl)-glycine, or beta-alanine
KIDVPWAGQYITSNPXIRFVSIDNKKRNIESSEIGP

SEQ ID NO: 3 - protein, 40 aa, Artificial Sequence
  Based on homo sapiens sequence
  MISC_FEATURE (7)..(7): X is glycine, serine, alanine, leucine, valine, isoleucine, norleucine, cyclopropyl-glycine, (cyclopropylmethyl)-glycine, or beta-alanine
YITSNPXIRFVSIDNKKRNIESSEIGPSLVIHTTVPFGVT

SEQ ID NO: 4 - protein, 27 aa, Artificial Sequence
  Based on homo sapiens sequence
  MISC_FEATURE (7)..(7): X is glycine, serine, alanine, leucine, valine, isoleucine, norleucine, cyclopropyl-glycine (cyclopropylmethyl)-glycine, or beta-alanine
YITSNPXIRFVSIDNKKRNIESSEIGP

SEQ ID NO: 5 - protein, 49 aa, Artificial Sequence
  Based on homo sapiens sequence
  MISC_FEATURE (16)..(16): X is glycine, serine, alanine, leucine, valine, isoleucine, norleucine, cyclopropyl-glycine, (cyclopropylmethyl)-glycine, or beta-alanine
KIDVPWAGQYITSNPXIRFVSIDNKKRNIESSEIGPSLVIHTTVPFGVT

SEQ ID NO: 6 - protein, 36 aa, Artificial Sequence
  Based on homo sapiens sequence
KIDVPWAGQYITSNPAIRFVSIDNKKRNIESSEIGP

SEQ ID NO: 7 - protein, 40 aa, Artificial Sequence
  Based on homo sapiens sequence
YITSNPAIRFVSIDNKKRNIESSEIGPSLVIHTTVPFGVT

SEQ ID NO: 8 - protein, 27 aa, Artificial Sequence
  Based on homo sapiens sequence
YITSNPAIRFVSIDNKKRNIESSEIGP

SEQ ID NO: 9 - protein, 49 aa, Artificial Sequence
  Based on homo sapiens sequence
KIDVPWAGQYITSNPAIRFVSIDNKKRNIESSEIGPSLVIHTTVPFGVT

SEQ ID NO: 10 - protein, 27 aa, Artificial Sequence
  Based on homo sapiens sequence
YITSNPVIRFVSIDNKKRNIESSEIGP

SEQ ID NO: 11 - protein, 40 aa, Artificial Sequence
  Based on homo sapiens sequence
YITSNPSIRFVSIDNKKRNIESSEIGPSLVIHTTVPFGVT

SEQ ID NO: 12 - protein, 36 aa, Artificial Sequence
  Based on homo sapiens sequence
  MISC_FEATURE (25)..(25): X is D-lysine
KIDVPWAGQYITSNPAIRFVSIDNXKRNIESSEIGP

SEQ ID NO: 13 - protein, 36 aa, Artificial Sequence
  Based on homo sapiens sequence
KIDVPWAGQYITSNPSIRFVSIDNKKRNIESSEIGP

SEQ ID NO: 14 - protein, 21 aa, Artificial Sequence
  Based on homo sapiens sequence
YITSNPAIRFVSIDNKKRNIE

SEQ ID NO: 15 - protein, 20 aa, Artificial Sequence
  Based on homo sapiens sequence
AIRFVSIDNKKRNIESSEIP

SEQ ID NO: 16 - protein, 36 aa, Artificial Sequence
  Based on homo sapiens sequence
KIDVPWAGQYITSNPGIRFVSIDNKKRNIESSEIGP

SEQ ID NO: 17 - protein, 40 aa, Artificial Sequence
  Based on  homo sapiens sequence
YITSNPGIRFVSIDNKKRNIESSEIGPSLVIHTTVPFGVT

SEQ ID NO: 18 - protein, 49 aa, Artificial Sequence; identical to SEQ ID NO: 5
  Based on homo sapiens sequence
  MISC_FEATURE (16)..(16): X is cyclopropyl-glycine

SEQ ID NO: 19 - protein, 27 aa, Artificial Sequence
  Based on homo sapiens sequence
  MISC_FEATURE (7)..(7): X is (cyclopropylmethyl)-glycine
YITSNPXIRFVSIDNKKRNIESSEIGP

SEQ ID NO: 20 - protein, 36 aa, Artificial Sequence; identical to SEQ ID NO: 6
  Based on homo sapiens sequence
  MISC_FEATURE (1)..(1): bisPEG5000 is attached to the N terminus of lysine